(19) United States
(12) Patent Application Publication
(10) Pub. No.: US 2002/0102717 A1
(43) Pub. Date: Aug. 1, 2002

(54) Refillable, reusable pregnancy test

(76) Inventor: Erika Lynn Hadeed, Stafford, VA (US)

Correspondence Address: Erika Lynn Hadeed, 911 Madison Court, Stafford, VA, 22554, US

(21) Appl. No.: 10/039,644

(22) Filed: Dec. 19, 2001

Related U.S. Application Data
(60) Provisional application No. 60/254,646, filed on Dec. 9, 2000.

(51) Int. Cl. C12M 1/34; G01N 33/543

(52) U.S. Cl. 435/287.2; 436/518

(57) ABSTRACT

This invention relates to an improved analytical device for collecting, analyzing, and storing biological samples and, primarily, to an analytical device used in the analysis of biological fluids such as urine.

### BACKGROUND-FIELD OF INVENTION

This invention relates to a novel analytical device for collecting, analyzing, and storing biological samples, and, more specifically, to an analytical device used in the analysis of biological fluids such as urine.

### BACKGROUND-DESCRIPTION OF PRIOR ART

The sampling and testing of biological fluids, such as urine, for the presence of analytes provides important information regarding various health related matters, including pregnancy and conception.

For an example, current test devices use an immunoassay for determining pregnancy or conception. At the heart of the immunoassay is a reagent, such as an antibody, that specifically reacts with an analyte to form a reaction complex The immunoassay also can include one or more separate detection reagents that react with the reaction product to facilitate detection of the reaction complex. The reaction complex can usually be detected by the unaided eye. An immunoassay can produce qualitative or semi-qualitative results.

Current pregnancy test devices assay for hormones associated with pregnancy, such as chorionic gonadotropin. Normally, the presence of human chorionic gonadotropin in urine is an indicator that a woman may be pregnant Such test devices obtain qualitative results indicating either the presence or absence of chorionic gonadotropin. Typically, a pregnancy immunoassay contains an antibody directed against chorionic gonadotropin and a separate detection antibody.

Conception test devices also assay for hormones associated with the ovarian cycle, such as luteinizing hormone. Luteinizing hormone is present normally in urine, but its concentration increases markedly during ovulation, the time at which a woman is most likely to conceive. The probability that a woman can conceive a child thus increases with increasing concentration of luteinizing hormone in the urine. Typically, a conception immunoassay contains an antibody directed against luteinizing hormone and a separate detection antibody.

Current test devices use various sample collection and analytical methods to detect an analyte in urine. For example, in one method urine is collected in a container and a measured urine volume transferred to a solution containing the immunoassay. The reaction product is detected in the resulting solution or as applied to a solid support. However, this method requires that the urine volume be accurately measured to insure the reaction product is not so diluted that it cannot be easily detected. In many situations, such as at-home testing, accurate measurement of urine volume is difficult perform and undesirable. Moreover, urine collection in a container and transfer to a test device generally is undesireable because of sanitary concerns and the potential for contamination.

In another method, the urine is collected in a container and transferred to a test device having an absorbent material that conducts or “wicks” the urine via capillary attraction to the immunoassay on a membrane. Typically, such a membrane immunoassay or absorbent material contains an antibody directed against the antigen of interest conjugated with a dye agent and an antibody directed to the antigen immobilized on the membrane immunoassay at a position “upstream” from the dye conjugate antibody. As the urine flows through the membrane immunoassay, the dye conjugate antibody binds the antigen and flows to the immobilized antibody where an antibody “sandwich” reaction complex is formed. A second absorbent material is positioned upstream from the immobilized antibody and in fluid flow contact with the membrane immunoassay to draw urine through the membrane immunoassay and collect urine and unbound dye conjugate antibody. The immobilized antibody typically is bound to the membrane in a line across the membrane immunoassay which results in a positive reaction appearing as a line.

A casing, usually made of plastic, surrounds the membrane immunoassay and the absorbent material in a manner that allows urine to be added only to the absorbent material. The absorbent material controls the volume of urine that contacts the immunoassay because only urine conducted by the absorbent material contacts the membrane immunoassay. Therefore, the volume of urine added to the absorbent material need not be accurately measured so long as a sufficient amount is added to allow the reaction to occur. An opening or window on one side of the casing over the membrane immunoassay permits the result to be observed. However, the collection of urine in a container and its transfer to the test device are disadvantages of the method because of sanitary concerns and the potential for contamination.

In another method, the urine is collected directly from the urine stream into the test device. The in-stream test device contains an absorbent material that is attached to and projects outward from a casing surrounding the above-described membrane immunoassay. The casing is designed as a handle to facilitate inserting the absorbent material into the stream during urination. The absorbent material is rigid so as to prevent being deflected out of the stream, which would further prevent the collection of a sufficient amount of urine. As described above, the absorbent material controls the amount of urine that contacts the membrane immunoassay, and an opening or window on the casing permits the result to be observed After collecting the urine, a cap may be placed over the absorbent material to contain the residual urine on the absorbent material and to facilitate handling.

In yet another method, the casing is designed in such a way that permits the casing to be reused while only the parts that come in contact with the urine and convey the test results are later disposed of In this method, a cassette, comprising of a first absorbent material for collecting the urine sample, a membrane immunoassay, and an optional second absorbent material, is inserted into the casing and removed from the casing through either an opening in the top or the bottom of the casing. This particular test device casing has a major disadvantage. The potential frequency of use and reuse of this devise by one or more individuals poses the likely threat of cross-contamination of test samples with prior test samples since there is no way to allow the inner chambers of this casing to be exposed for proper and thorough cleansing, which is necessary to protect the integrity of the test sample and the ensuing result.

A disadvantage of other current test devices is that they are expensive to use because each test device can only be used once. Since the cost of each test device is relatively high, a user incurs significant expense when multiple tests are required. For example, a conception test often requires testing for luteinizing hormone once a day for five or more days to optimize the probability of conception. In another example, infertile couples require frequent pregnancy tests to verify the efficacy of various fertility treatments. The high cost of current test devices is a disadvantage particularly in less wealthy regions of the world where the need for such test devices is great but cost significantly limits their use.

All current disposable tests also add the burden of extra waste to the earth's already fragile and potentially compromised ecosystems.

There thus is a need for a test device that detects analytes in urine which is more economical to manufacture and use than current test devices. There is also a need for a reusable test device that allows the casing interior to be thoroughly cleaned prior to refilling the test materials and reusing the test device. Finally, the test device should be simple enough to be used by the lay individual outside of a medical facility or any location such that medically trained individuals are not required to use the device. For instance, pregnancy tests are often conducted by the patient in her own home. The present invention satisfies these needs and provides related advantages as well.

### SUMMARY

This invention provides an analytical device for collecting and analyzing biological fluids, especially urine, which comprises an absorbent material, an assay on a support means, and a casing that can easily and simply be opened for the thorough cleansing of its interior.

### OBJECTS AND ADVANTAGES

Accordingly, besides the objects and advantages of the analytical device described above, some objects and advantages of the present invention are:

(a) to provide a casing that includes a fluid constriction flange of a size that limits the flow of fluid to the membrane immunoassay from the absorption material when the casing is closed and keeps the membrane immunoassay and absorption material in contact with each other as the test is being conducted;

(b) to provide at least one turning joint along one or more sides of the casing to allow access to the interior cavity of the casing for the ease of thoroughly cleaning and drying the components and allowing the test materials to be simply inserted; and

(c) to provide at least one latch for opening and closing the casing.

Still further objects and advantages will become apparent from a consideration of the ensuing description and drawings.

### DRAWING FIGURES

In the drawings, closely related figures have the same number but different alphabetic suffixes.

FIGS. 1 and 2 are a front and side view of a cap, respectively.

FIGS. 3A and 3B are a side and front view of a casing, respectively.

FIG. 3C is an exploded view of a latch os the casing of FIG. 3B, FIG. 4B and FIG. 5.

FIG. 3D is an inside view of a casing and shows a membrane immunoassay on a support means and an absorbent material fully inserted into the casing.

FIGS. 4A and 4B are a side and front view of a casing, respectively.

FIG. 5 is a front view of a casing .

FIG. 6A and 6B are a side and front view of a casing, respectively.

FIG. 6C is an inside view of a casing and shows a membrane immunoassay on a support means and an absorbent material fuilly inserted into the casing.

FIG. 6D shows a top view of a casing in FIG. 6B.

FIGS. 6E and 6F are cut away views of the casing in FIG. 6B. FIG. 6E shows the cross-section of the latch mechanism in an open position. FIG. 6F shows the cross-section of the latch mechanism in a closed position.

FIGS. 7A and 7B are a front and side view of an absorbent material, respectively.

FIGS. 8A and 8B are a front and side view of a membrane immunoassay on a support means.

FIG. 9 is an inside view of the casing's lower chamber in FIGS. 3C and 6C showing a channel.

| | |
|---|---|
| 10 cap | 12 casing |
| 14 viewing areas | 16 turning joint |
| 18 latch | 20 membrane immunoassay |
| 22 first absorbent material | 24 latch holder |
| 26 channel | 28 fluid constriction flange |
| 30 support means | 32 latch release grip |
| 34 optional second absorbent material | |

### DESCRIPTION

This invention provides an analytical device for collecting and analyzing biological fluids, especially urine, and comprises an absorbent material and an assay that can be easily inserted into and removed from a casing. The casing can be easily and fully opened for the thorough cleansing of its top and lower chambers.

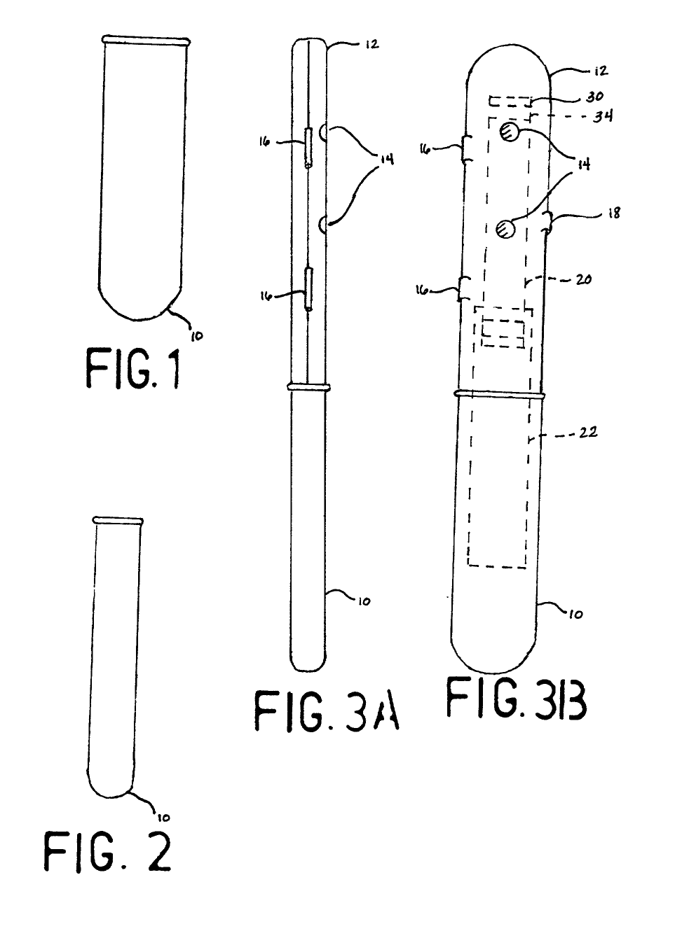

FIGS. 1-3D-Preferred Embodiment

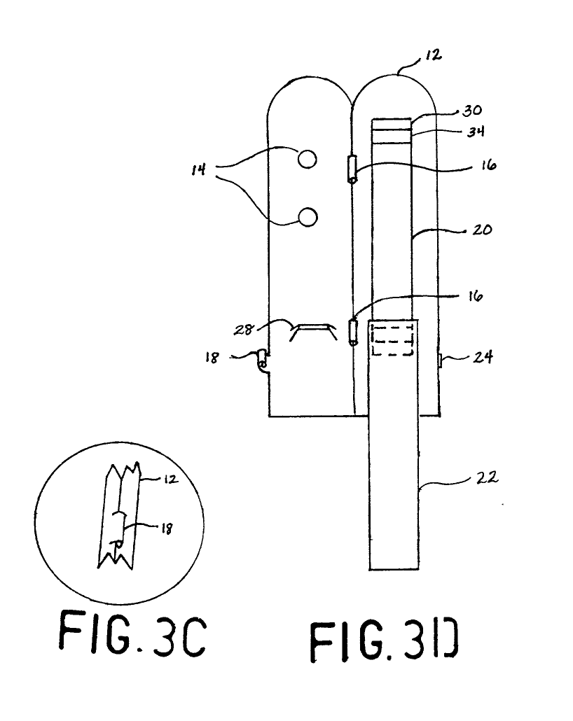

A preferred embodiment of the present device is illustrated in FIG. 3B, which shows a casing 12 having two turning joints 16, a latch 18, a viewing area 14, and a cap 10, as seen in FIGS. 1 and 2. FIG. 3A shows turning joints 16 on one side of casing 12 of FIG. 3B. FIG. 3C is an exploded view of latch 18 on the side opposite that with turning joints 16 on casing 12 of FIG. 3B.

In a preferred embodiment, casing 12 is made of any moisture impermeable material including, for example, plastics such as polystyrene, polyproplyene, polyvinylchloride and acrylic. The casing can be made by means known in the art appropriate for the material. For example, a casing made of plastic can be machined or molded, including injection and vacuum molded.

The “casing” is made of two parts, the upper and lower chambers. The parts are attached together at turning joints 16. A “turning joint” can be formed by molding or welding the plastic material of the casing so that the two chambers are firmly attached to each other at the turning joint. A turning joint can also be made of a material separate from the casing, such as metal.

A “latch” 18 can be welded or molded from the same material of the upper chamber of the casing. The latch can also be made from a separate material, such as metal.

A “viewing area” 14 can be one or more openings on a casing's outer surface on the upper chamber. The viewing area is covered by a separate window part or a transparent casing in order to protect analytical materials from possible urine spray and droplets. The viewing area allows the results of a test to be seen.

A “cap” 10 has an open and closed end and forms a tight fit with the casing by any known means including, for example, a snap fit, friction fit, and mechanical fastening.

FIG. 3D shows a casing open on turning joints 16. The inside of the upper chamber shows a viewing area 14, a fluid constriction flange 28, and a latch 18.

The “fluid constriction flange” 28 is a structure designed to limit fluid flow by means of a friction fit. The fluid constriction flange can be molded or welded from the material of the upper chamber of the casing.

The inside of the lower chamber in FIG. 3D shows a support means 30 attached to a membrane immunoassay 20, an optional second absorbent material 34, a first absorbent material 22, and a latch holder 24. A channel 26 is a formation in the cavity of the lower chamber of casing 12 and is illustrated in FIG. 9.

A “membrane immunoassay” 20 has a first and second end and comprises at least one reagent that forms a visible reaction complex indicating the presence of an analyte and a porous carrier capable of wicking aqueous fluid. The reaction complex can be read through a viewing area 14.

Any reagent can be used in any known format such as, for example, sandwich and competetive binding formats, to specifically detect an analyte in a biological fluid such that a visible reaction complex is formed. Examples of such reagents are those disclosed in H. J. Friesen, U.S. Pat. No. 4,861,711, issued Aug. 29, 1989; J. Bunting, U.S. Pat. No. 4,271,140, issued Jun. 2, 1981; European Patent Publication No. 0 284 232 and European Patent Publication No. 0 291 194. Such reagents can form a visible reaction complex with analytes such as, for example, hormones, proteins, haptens, immunoglobulin, polynucleotides, steroids, drugs, infectious disease agents (bacterial or viral) such as Streptococcus, Neisseria, and Chiamydia.

Preferred reagents include antibodies to a hormone or infectious disease agent. Such antibodies include mobile antibodies conjugated to a signal agent or immobilized antibodies on the membrane. Mobile conjugated antibodies can be impregnated into the membrane immunoassay. The mobile conjugated antibodies are located downstream from a zone on the membrane immunoassay that contains immobilized antibodies. The mobile color conjugated antibodies bind to the hormone or infectious disease agent and are carried to the zone containing the immobilized antibodies where a sandwich antibody-hormone complex is formed and visualized. Preferred antibodies include anti-human chorionic gonadotropin antibodies and anti-human luteinizing hormone antibodies, especially murine monoclonal antibodies and especially those that have been affinity purified Preferred signal agents include colored latex spheres and colloidal metals. Such reagents and signal agents include those disclosed by D. Yost et al. U.S. Pat. No. 4,954,452, issued Sep. 4, 1990; J Leuvering U.S. Pat. No. 4,313,734, issued Feb. 2, 1982; P. Tarcha et al. U.S. Pat No. 5,252,459, issued Oct. 12, 1993; T. Gribman et al. U.S. Pat. No. 4,373,932, issued Feb. 15, 1983; and R. Campbell, U.S. Pat No. 4,703,013, issued Oct. 27, 1987.

The porous carrier of the membrane immunoassay is any bibulous, porous, or fibrous material capable of rapidly absorbing an aqueous fluid and conducting the fluid via capillary attraction. Suitable materials are described, for example, in H. J. Friesen, U.S. Pat. No. 4,861,711, issued Aug. 29, 1989; J. Bunting, U.S. Pat No. 4,271,140, issued Jun. 2, 1981; European Patent Publication No. 0 284 232 and European Patent Publication No. 0 291 194. Preferred porous materials include nitrocellulose, nylon, paper, and silica gel. An advantage of a nitrocellulose membrane is that an immobilized antibody described above can be attached without prior chemical treatment. However, antibodies can be immobilized on other materials such as filler paper using well known chemical coupling methods such as, for example, CNBr carbonyldimidazole or tresyl chloride.

The membrane immunoassay can be any size compatible with a casing that allows a reaction complex to be visualized.

The membrane immunoassay can be a single layer or a multilayered membrane immunoassay so long as it forms a fluid flow contact with a first absorbent material 22 and an optional second absorbent material 34, when present. For example, two or more membranes may be completely or partially layered over each other and each contain different reagents. In another example, a lower layer can facilitate attachment of an upper layer membrane immunoassay to a support means 30.

The membrane immunoassay also can be one continuous membrane immunoassay or several membranes connected in series. For example, two membranes can be connected end-to-end and each contain different reagents.

A “support means” 30 is any material to which the membrane and an optional second absorbent material can be attached to form a rigid, semi-rigid, or flexible strip format. Preferred materials include polystyrene, polyproplyene, acrylic, and polyvinyl chloride plastics.

An “optional second absorbent material” 34 is any bibulous, porous, or fibrous material capable of rapidly absorbing an aqueous fluid and conducting the fluid via capillary attraction. Suitable materials are described, for example, in H. J. Friesen, U.S. Pat. No. 4,861,711, issued Aug. 29, 1989; J. Bunting, U.S. Pat. No. 4,271,140, issued Jun. 2, 1981; European Patent Publication No. 0 284 232 and European Patent Publication No. 0 291 194. Preferred materials include paper, nitrocellulose, nylon, and silica gel.

The optional second absorbent material can be any size compatible with a support means so long as it aids capillary flow through the membrane immunoassay. The second absorbent material can be a single layer or multilayered so long as the second absorbent material forms a fluid contact with the membrane immunoassay. The second absorbent material can also be a desiccant such as, for example, silica gel.

A “first absorbent material” 22 is any bibulous, porous, or fibrous material capable of rapidly absorbing an aqueous fluid and conducting the fluid via capillary attraction. Examples of such materials are those disclosed in H. J. Friesen, U.S. Pat. No. 4,861,711, issued Aug. 29, 1989; J. Bunting, U.S. Pat. No. 4,271,140, issued Jun. 2, 1981; European Patent Publication No. 0 284 232 and European Patent Publication No. 0 291 194. Preferred materials include paper, nitrocellulose, nylon, and silica gel.

The first absorbent material optionally can also comprise a mobile reagent that specifically binds to an analyte in the fluid to form a visible reactive complex with a reagent in the membrane immimoassay. Examples of such reagents are those disclosed in the above-mentioned patents and applications. As discussed in the detailed description of the membrane immunoassay, a preferred reagent is a mobile antibody directed against chorionic gonadotropin or luteinizing hormone conjugated to a colored latex sphere or colloidal metal.

The first absorbent material can be any size compatible with a casing so long as it is of a sufficient size to transport a sufficient amount of fluid to the membrane immunoassay in order to detect the analyte with the reagent(s) on the membrane. The first absorbent material can be a single layer or multilayered absorbent material so long as the first absorbent material, aided by a fluid constriction flange 28, is in fluid flow contact with the membrane immunoassay. The first absorbent material can be any length so long as a cap 10 is able to fit over the first absorbent material, forming a tight fit with the casing and facilitating the handling of a sample.

A “latch holder” 24 is any structure that allows a latch 18 to be anchored firmly to the lower chamber of a casing 12 when the casing is in a closed position. A preferred material is plastic molded from the lower chamber of the casing itself A holder could also be made of alternative materials, such as metal, welded to the casing to provide an anchor for a latch.

A “channel” 26 is a recess in the cavity of the lower chamber of a casing 12 for the purpose of providing a housing for a membrane immunoassay 20 and a first absorbent material 22. The channel must be a size sufficient to secure the membrane and the first absorbent material within the lower chamber of the casing, aided by a fluid constriction flange 28, in order to maintain a path for conducting fluid flow through the first absorbent material to the membrane.

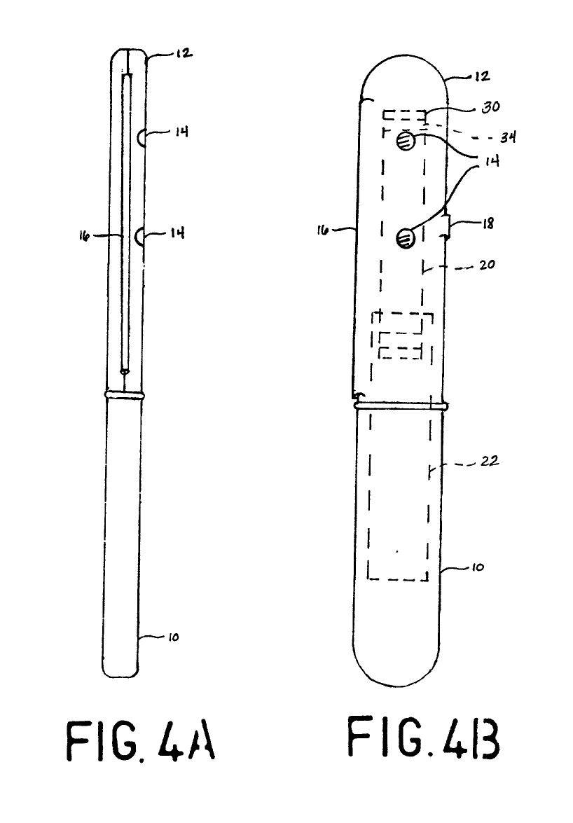

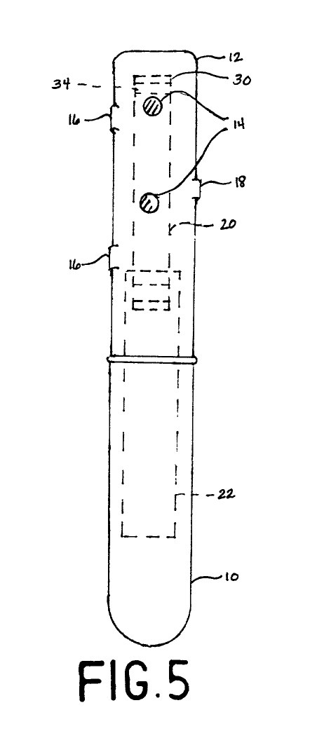

FIGS. 4A-5-Additional Embodiments

Additional embodiments are shown in FIGS. 4A, 4B, and 5. In FIGS. 4A and 4B a casing 12 has only one turning joint 16 measuring approximately the length of the casing; in FIG. 5 the casing has a more squared shape at the top as opposed to the rounded casing in the preferred embodiment of FIG. 3B.

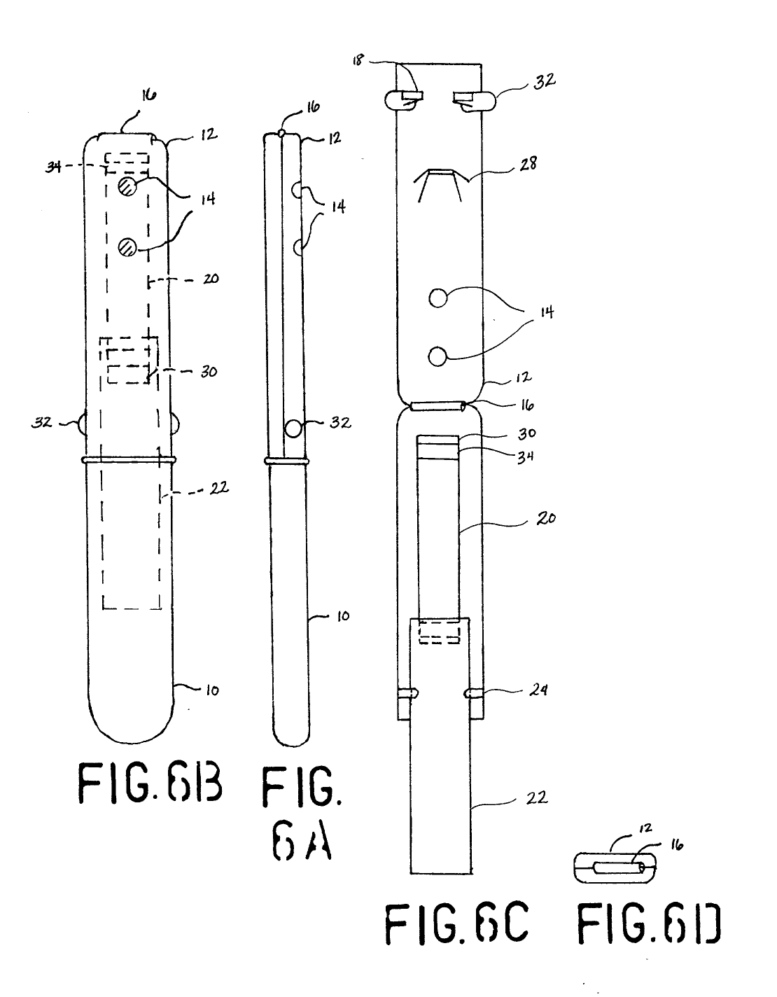

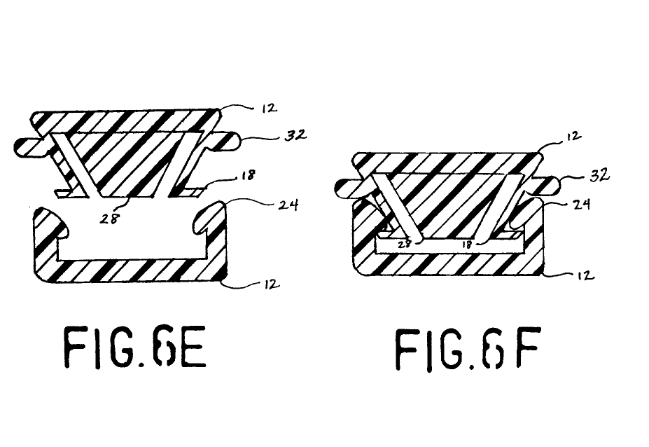

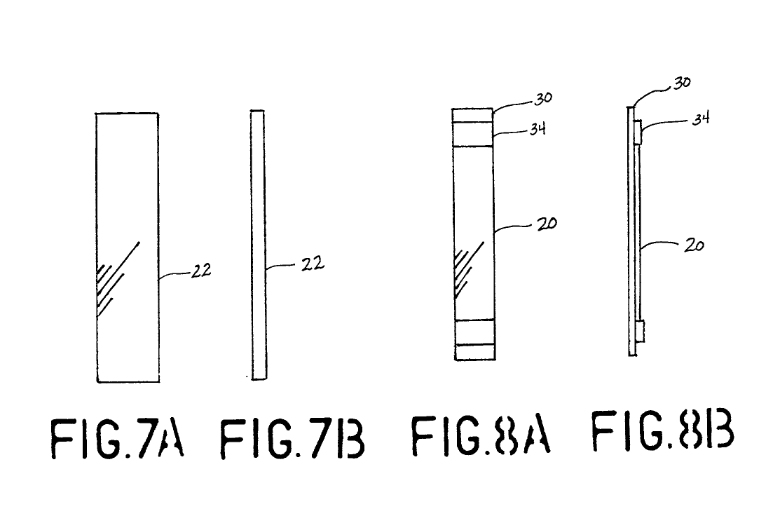

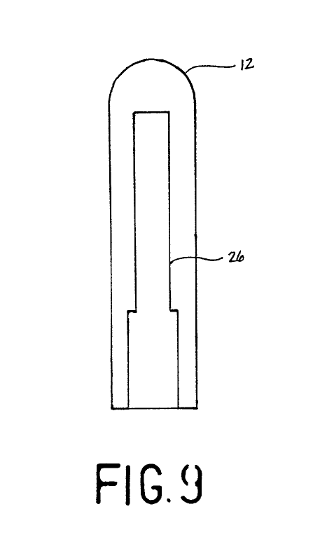

FIGS. 6A-6F-Alternative Embodiments

Another configuration is one where a casing 12 opens on a turning joint 16 located at the top of the casing; FIGS. 6A, 6B, and 6C show a turning joint 16 positioned in such a way that the upper and lower chambers of the casing are attached via the tuningjoint. FIGS. 6A, 6B, and 6C also show a latch release grip 32 on both sides of the upper chamber of the casing; FIG. 6C shows an internal perspective of the casing. The upper chamber reveals a latch 18 connected to a latch release grip 32. Within the cavity of the lower chamber on either side is a latch holder 24. A channel 26 is recessed into the lower chamber as illustrated in FIG. 9. FIG. 6D shows a turning joint 16 attached to the top of a casing 12, connecting the upper and lower chambers of the casing; FIG. 6E is a cross-section of the casing in FIG. 6B that illustrates the upper and lower chambers in a disconnected position and reveals the relationship of the latch release grip 32, the latch 18, and the fluid constriction flange 28 to the latch holders 24; FIG. 6F is a cross-section of the casing in FIG. 6B that illustrates the upper and lower chambers in a connected position via latch 18 and the latch holder 24.

### ADVANTAGES

There are a number of advantages to my analytical device:

(a) After the purchase of the initial test device kit, the consumer need only refill the casing with the first absorbent material and the membrane immunoassay attached to a support means instead of throwing away all of the components of the kit after a single use. This will eliminate the extra cost incurred from disposable kits, which require much more material to manufacture and package.

(b) Less material used in manufacturing and packaging means less waste in the environment.

(c) The casing's capacity to be opened and closed allows the consumer to thoroughly clean the cavities of the chambers of the casing in order to remove all traces of an analyte and avoid cross-contaminating future tests. This is especially important since many women may choose to share the device or loan it to others due to the convenience of the reusable and refillable properties of the device. The analytical device described in H. Noda, U.S. Pat. No. 5,900,379, issued May 4, 1999 fails to address the important need for providing a way to thoroughly clean a casing's interior after each use in order to protect the integrity of future tests.

(d) The casing's turning joint feature allows fast and easy access to a casing's a interior regions.

(e) If there is ever any reason to preserve a test result, the support means containing the immunoassay can be stored and preserved in a separate and appropriate container. The casing can be free, therefore, to refill and reuse almost immediately.

Operation-FIGS. 3, 4, 5, 6

The manner of using the analytical device is similar to comparable devices in present use. The following example illustrates collecting an in-stream urine sample and detecting the presence of chorionic gonadotropin using the analytical device illustrated in FIGS. 3, 4, and 5. The process begins by releasing latch 18 in FIGS. 3, 4, and 5 from latch holder 24. The upper chamber of casing 12 is opened on turning joints 16 to a reach of about 180 degrees. The cavities of the chambers of the casing are now exposed.

A membrane immunoassay 20 attached to a support means 30 is selected and placed in channel 26. A first absorbent material 22 is then placed in channel 26 such that one end covers the lower portion of the membrane immunoassay, as seen in FIG. 3D. Next, the upper chamber is closed upon the lower chamber and is secured when latch 18 is anchored to latch holder 24. The assembled device is inspected to determine that the first absorbent material extends beyond the casing and the membrane immunoassay can be seen through viewing area 14. FIG. 3B shows all parts in correct registration inside the casing.

Cotlventional in-stream urine collection procedures are then used to collect a sample. A sample can be collected at any time of day, but for best results, it is best to test the first urine of the morning because it contains the highest concentration of ehorionic gonadotropin. After urination has started, the first absorbent material of the analytical device is inserted into the urine stream until the entire first absorbent material is wet, about five seconds.

The analytical device is removed from the urine stream with the first absorbent material end pointing downward Optionally, a cap 10 may be placed over the first absorbent material in order to facilitate the handling of the sample.

The result is obtained usually in about two to five minutes and appears in the viewing area. Any indication of a positive reaction indicates pregnancy.

After the analysis, the first absorbent material and the support means containing a membrane immunoassay can be removed from the casing. The casing can then be washed thoroughly and reserved for a subsequent analysis. The result can be discarded or stored as desired.

The analytical procedures for detecting the presence of chorionic gonadotropin or other analytes are the same for the device in FIG. 6. The casing, however, as seen in FIGS. 6A, 6B, and 6C has a different method of operability than the casing of FIGS. 3, 4, and 5. To open the casing, firmly press latch release grip 32 on both sides of the upper chamber in order to release latch 18 from latch holder 24 and lift the upper chamber on turning joint 16 away from the lower chamber. As shown in FIG. 6C, the appropriate materials are inserted to perform the above-mentioned urine analysis to detect an analyte. Next, the upper chamber of casing 12 is closed upon the lower chamber and is secured when latch 18 is anchored to latch holder 24. The analysis can then be conducted and the results observed.

Conclusion, Ramifications, and Scope

Accordingly, the reader will see that the analytical device as represented can be easily used by the average consumer with the option to reuse the casing in future analyses. In addition, the casing can be opened to completely expose the cavities of the upper and lower chambers for a necessary and thorough cleaning between each use. This is of great importance since many women may choose to share or loan the device to others, potentially increasing an occurrence of cross-contamination. Furthermore, the analytical device has additional advantages in that

it permits women who may be experiencing infertility to screen for ovulation and pregnancy as needed without having to purchase a complete test kit for each analysis;

it permits manufacturers to use less material in overall production;

it helps reduce waste incurred from the excessive packaging of disposable test kits, which is beneficial to the environment;

it provides a casing of various sizes, colors, and designs that opens to completely expose the cavitiy of the chambers for assembling the analytical materials and for thoroughly cleansing the device of any residual matter from a previous analysis.

Although the device has been described with reference to the figures and examples, provided above, it should be understood that various modifications can be made by those skilled in the art without departing from the device.

Thus the scope of the invention should be determined by the claims and their legal equivalents, rather than by the examples given.

I claim:

1. An analytical device comprising:
a) a first absorbent material;
an optional second absorbent material;
a membrane immunoassay having a first end and a second end comprising at least one immobilized reagent that forms a visible reaction complex indicating the presence of an analyte in fluid and a porous carrier that wicks aqueous fluid;
a support means, wherein:
said membrane immunoassay is arranged on and attached to said support means; and
said first end of said membrane immunoassay is in fluid flow contact with said first absorbent material, and said second end of said membrane immunoassay is in fluid flow contact with said optional second absorbent material; and
b) a casing including:
a fluid constriction flange;
a channel that is the size of or larger than said first absorbent material and said support means;
a viewing area;
at least one turning joint; and
at least one latch comprising a latch holder and a latch release grip; and
c) a cap having an opened end and a closed end, providing a cover for said first absorbent material and forming a tight fit with said casing.

2. The analytical device of claim 1 wherein said immobilized reagent that forms the visible reaction in said membrane immunoassay is an immobilized antibody.

3. The analytical device of claim 2 wherein said fluid constriction flange extends into said casing forming a contact point for said first absorbent material and the membrane assay.

4. The analytical device of claim 3 wherein said channel is present and forms a housing with a raised edge in which to place said first absorbent material and the membrane assay.

5. The analytical device of claim 4 wherein said turning joints form an attachment on at least one side of said casing providing an opening means for exposing the inside of said casing.

6. The analytical device of claim 5 wherein said latch provides a means for releasing said casing to an open position and for securing said casing to a closed position.
